(19) United States
(12) Patent Application Publication
(10) Pub. No.: US 2026/0049051 A1
(43) Pub. Date: Feb. 19, 2026

(54) DIASTEREOMER PREPARATION METHOD, AND SOLID-PHASE PHOTOSENSITIZER

(71) Applicants: TOKYO UNIVERSITY OF SCIENCE FOUNDATION, Tokyo (JP); IWASAKI ELECTRIC CO., LTD., Toyko (JP)

(72) Inventors: Hideyo TAKAHASHI, Tokyo (JP); Kosho MAKINO, Tokyo (JP); Mayuko SUGA, Tokyo (JP); Kunio KANEMARU, Tokyo (JP); Noritaka KURODA, Kyoto-shi, Kyoto (JP); Eigo MUTO, Kyoto-shi, Kyoto (JP); Ryo SHIROMA, Kyoto-shi, Kyoto (JP)

(21) Appl. No.: 19/103,386

(22) PCT Filed: Aug. 25, 2023
(86) PCT No.: PCT/JP2023/030673

§ 371 Date: Feb. 12, 2025

(30) Foreign Application Priority Data
Sep. 1, 2022 (JP) 2022-139583

(51) Int. Cl. C07C 231/12; C07C 231/24; C07D 335/16

(52) U.S. Cl. CPC C07C 231/12; C07C 231/24; C07D 335/16

(57) ABSTRACT

A diastereomer preparation method that produces a desired diastereomer from a diastereomer mixture with high efficiency; and a solid-phase photosensitizer which is used in the method. In the method, one diastereomer among cis-trans isomers is selectively prepared utilizing recycle HPLC. The method includes a step A that is a separation step for introducing a cis-trans isomer mixture into the separation column to selectively collect one diastereomer; a step B that is a photoisomerization reaction step for irradiating a solution containing the other diastereomer which is obtained after the step A or C with light in the presence of a photosensitizer in a photoisomerization reactor to induce a photoisomerization reaction; and a step C that is a step for introducing a cis-trans isomer mixture produced in the step B into a separation column to selectively collect the one diastereomer.

### TECHNICAL FIELD

The present invention relates to a diastereomer preparation method and a solid-phase photosensitizer.

### BACKGROUND ART

Conventionally, it has been known that isomers have different properties from each other. For example, diastereomers of alkenes (E and Z isomers in cis-trans isomers) generated in the olefination reaction of carbonyl compounds have different physical properties. For example, in a pharmaceutical which has an alkene skeleton, it is desirable to obtain one isomer in high purity in a clinical trial and in a pharmaceutical manufacturing stage.

In the background described above, various synthesis methods for selectively preparing one diastereomer have been proposed (see, for example, Non-Patent Documents 1 and 2).

However, it is nearly impossible to selectively prepare only one diastereomer. Although diastereomers have also been separated by chromatography, the remaining diastereomer has been wasted.

### CITATION LIST

### Non-Patent Document

Non-Patent Document 1: Y. Zhao et al., J. Am. Chem. Soc. 2015, 137, pp. 5199-5203
Non-Patent Document 2: Tetsuya Ezawa and six others, “Development of Diastereoconvergent (3+2) Cycloaddition Reactions”, [online], Jun. 10, 2021, RIKEN, [retrieved on Aug. 19, 2022], Internet <URL: https://www.riken.jp/press/2021/20210610 3/index.html>

### DISCLOSURE OF THE INVENTION

### Problems to be Solved by the Invention

An object of the present invention is to provide a diastereomer preparation method capable of obtaining a desired diastereomer in high yield from a diastereomer mixture and a solid-phase photosensitizer used in the diastereomer preparation method.

### Means for Solving the Problems

Specific means for achieving the object described above include the following embodiments.

<1> A diastereomer preparation method for selectively preparing one diastereomer of cis-trans isomers by utilizing recycling HPLC capable of continuously circulating through a separation column that separates the cis-trans isomers and a photoisomerization reactor that induces a photoisomerization reaction, the diastereomer preparation method including:
step A: a separation step of introducing a cis-trans isomer mixture into the separation column to isolate the one diastereomer;
step B: a photoisomerization reaction step of applying light to a solution containing the other diastereomer after the step A or step C in presence of a photosensitizer in the photoisomerization reactor to induce the photoisomerization reaction; and
step C: a step of introducing the cis-trans isomer mixture generated in the step B into the separation column to isolate the one diastereomer, in which the photosensitizer includes a thioxanthone skeleton represented by formula (1) below.

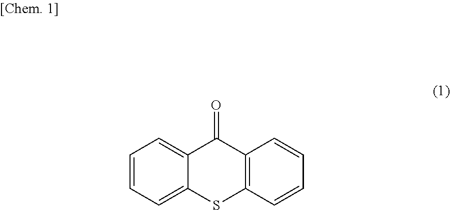 [SMILES: O=c1c2ccccc2sc2ccccc12]

<2> The diastereomer preparation method according to <1>, in which the photosensitizer is a solid-phase photosensitizer in which a structure represented by formula (2) below is covalently solid-phased to a carrier in the photoisomerization reactor via a linker.

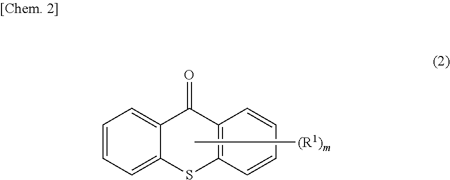

[In formula (2), R¹ represents an alkyl group having 2 to 3 carbon atoms, a hydroxy group or an alkoxycarbonyl group, m represents an integer from 0 to 5, and when m is an integer from 2 to 5, a plurality of Ris may be the same as or different from each other.]

<3> The diastereomer preparation method according to <2>, where the linker includes an alkylene group, an arylene group, an —NR—C(O)— group (R independently represents a hydrogen atom, an alkyl group, or a substituted alkyl group), or a functional group including a combination thereof.

<4> The diastereomer preparation method according to <3>, in which the solid-phase photosensitizer includes a structure represented by formula (3) below.

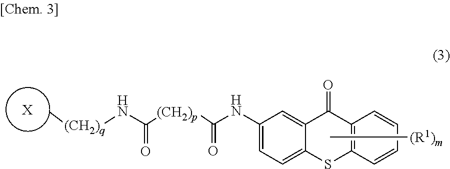

[In formula (3), R¹ and m are as defined in formula (2) above, p represents an integer from 1 to 10, q represents an integer from 2 to 10, and X represents a carrier.]

<5> The diastereomer preparation method according to <1>, in which the photosensitizer is represented by formula (4) below.

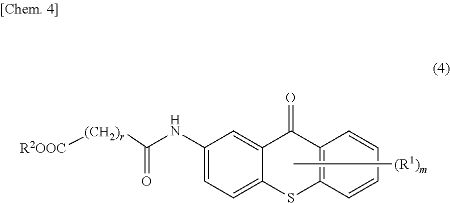

[In formula (4), R¹ represents an alkyl group having 2 to 3 carbon atoms, a hydroxy group, or an alkoxycarbonyl group, m represents an integer from 0 to 5, and when m is an integer from 2 to 5, a plurality of R's may be the same as or different from each other,
r represents an integer from 1 to 7, and
R² represents a hydrogen atom or an alkyl group having 1 to 4 carbon atoms.)

<6> The diastereomer preparation method according to <1>including step D below in a stage preceding the step A above: step D: a photoisomerization reaction step of applying light to a solution containing only the other diastereomer of the cis-trans isomers in presence of the photosensitizer in the photoisomerization reactor to induce the photoisomerization reaction, and thereby generating a cis-trans isomer mixture.

<7> The diastereomer preparation method according to <2>, in which a material of the carrier is silica, glass, a polymer material, or a composite material thereof.

<8> The diastereomer preparation method according to <1>, in which the cis-trans isomer is a cis-trans isomer of an alkene including an amide group in a molecule.

<9> The diastereomer preparation method according to <8>, in which the alkene including an amide group in the molecule is a Weinreb amide.

<10>A solid-phase photosensitizer including a structure represented by formula (3) below.

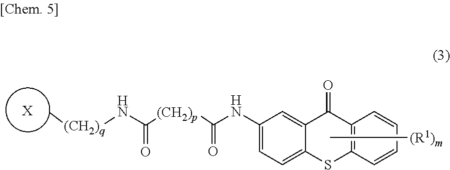

[In formula (3), R¹ represents an alkyl group having 2 to 3 carbon atoms, a hydroxy group, or an alkoxycarbonyl group, m represents an integer from 0 to 5, and when m is an integer from 2 to 5, a plurality of Ris may be the same as or different from each other,
p represents an integer from 1 to 10, q represents an integer from 2 to 10, and X represents a carrier.]

<11>A method for manufacturing a solid-phase photosensitizer including a structure represented by formula (3) below, the method including:

a step of reacting a compound represented by formula (5) below with a dicarboxylic acid represented by formula (6) below or an anhydride thereof to obtain a compound represented by formula (7) below; and

a step of reacting a carrier including a structure represented by formula (8) below with a compound represented by formula (7) below to obtain a solid-phase photosensitizer including a structure represented by formula (3) below.

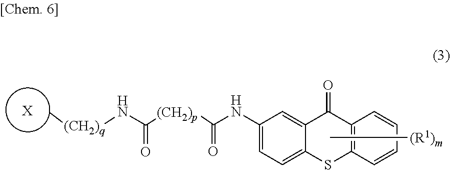

[In formula (3), R¹ represents an alkyl group having 2 to 3 carbon atoms, a hydroxy group, or an alkoxycarbonyl group, m represents an integer from 0 to 5, and when m is an integer from 2 to 5, a plurality of R's may be the same as or different from each other,
p represents an integer from 1 to 10, q represents an integer from 2 to 10, and
X represents a carrier.]

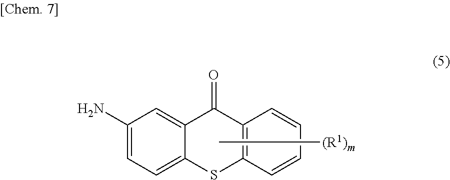

[In formula (5), R¹ and m are as defined in formula (3) above . . . ]

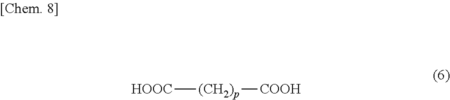

[In formula (6), p is as defined in formula (3) above.]

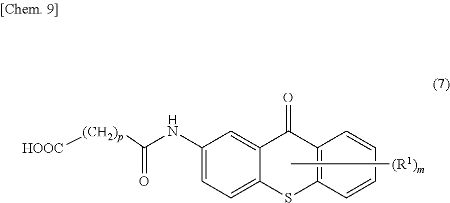

[In formula (7), R¹, m and p are as defined in formula (3) above.]

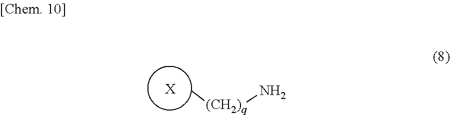

[In formula (8), q and X are as defined in formula (3) above.]

<12>A compound represented by formula (4) below.

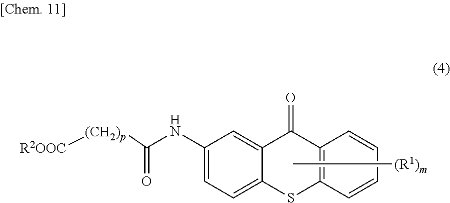

[In formula (4), R¹ represents an alkyl group having 2 to 3 carbon atoms, a hydroxy group, or an alkoxycarbonyl group, m represents an integer from 0 to 5, and when m is an integer from 2 to 5, a plurality of R's may be the same as or different from each other,
r represents an integer from 1 to 7, and
R² represents a hydrogen atom or an alkyl group having 1 to 4 carbon atoms.]

### Effects of the Invention

According to the present invention, it is possible to provide a diastereomer preparation method capable of obtaining a desired diastereomer in high yield from a diastereomer mixture and a solid-phase photosensitizer used in the diastereomer preparation method.

### BRIEF DESCRIPTION OF THE DRAWINGS

FIG. 1 is a diagram showing an example of a schematic configuration of recycling HPLC according to the present embodiment;

FIG. 2 is a diagram showing respective changes over time in the abundance ratio of the other diastereomer when a photoisomerization reaction is performed using thioxanthone alone as a photosensitizer and when a photoisomerization reaction is performed using a solid-phase photosensitizer obtained by covalently binding thioxanthone to a carrier;

FIG. 3 is a diagram showing changes over time in the abundance ratio of the other diastereomer when a leaching test is performed using thioxanthone alone as a photosensitizer and when a leaching test is performed using a solid-phase photosensitizer obtained by covalently binding thioxanthone to a carrier; and

FIG. 4 is a diagram showing changes over time in the intensity of isomer detection performed by a diastereomer preparation system according to the present embodiment.

### PREFERRED MODE FOR CARRYING OUT THE INVENTION

### Diastereomer Preparation Method

A diastereomer preparation method according to the present embodiment is a diastereomer preparation method for selectively preparing one diastereomer of cis-trans isomers by utilizing recycling HPLC capable of continuously circulating through a separation column that separates the cis-trans isomers and a photoisomerization reactor that induces a photoisomerization reaction, and the diastereomer preparation method includes steps A to C below:

step A: a separation step of introducing a cis-trans isomer mixture into the separation column to isolate the one diastereomer;

step B: a photoisomerization reaction step of applying light to a solution containing the other diastereomer after the step A or step C in the presence of a photosensitizer in the photoisomerization reactor to induce the photoisomerization reaction; and

step C: a step of introducing the cis-trans isomer mixture generated in the step B into the separation column to isolate the one diastereomer.

The diastereomer preparation method according to the present embodiment may include, in addition to the steps A to C described above, step D below in the preceding stage:

step D: a photoisomerization reaction step of applying light to a solution containing only the other diastereomer of the cis-trans isomers in presence of a photosensitizer in the photoisomerization reactor to induce the photoisomerization reaction, and thereby generating a cis-trans isomer mixture.

(Cis-trans isomer mixture)

The cis-trans isomer mixture is not particularly limited as long as the cis-trans isomer mixture is a compound in which E and 2 cis-trans isomers defined by IUPAC based on atoms, bonds or planes in a chemical structure are present.

Examples of the preferred cis-trans isomer mixture include an alkene including an amide group in the molecule. Examples of the alkene including an amide group in the molecule include: a Weinreb amide represented by formula (9) below; a cinnamamide represented by formula (10) below; and the like. The cis-trans isomer mixture is not limited to the compounds described above.

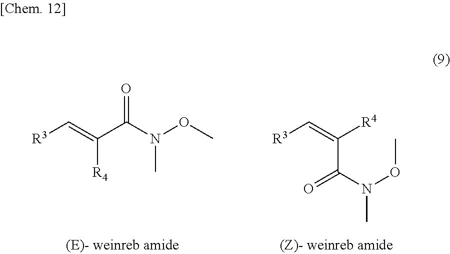

[In formula (9), R³ represents a halogen atom, an alkyl group which may include a substituent, an alkenyl group which may include a substituent, an alkynyl group which may include a substituent, an aryl group which may include a substituent, a heteroaryl group which may include a substituent, an arylalkyl group which may include a substituent or a heteroarylalkyl group which may include a substituent, and R⁴ represents a hydrogen atom or an alkyl group.]

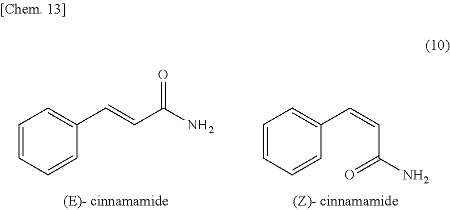 [SMILES: NC(=O)/C=C/c1ccccc1 ; NC(=O)/C=C\c1ccccc1]

In formula (9) described above, examples of the halogen atom represented by R³ include a fluorine atom, a chlorine atom, a bromine atom, an iodine atom and the like.

In formula (9) described above, examples of the alkyl group represented by R³ or R⁴ include linear, branched or cyclic alkyl groups having 1 to 20 carbon atoms such as a methyl group, an ethyl group, an n-propyl group, an isopropyl group, an n-butyl group, an isobutyl group, a sec-butyl group, a text-butyl group, an n-pentyl group, an isopentyl group, a cyclopentyl group, an n-hexyl group, a cyclohexyl group and the Like.

In formula (9) described above, examples of the alkenyl group represented by R³ include linear or branched alkenyl groups having 2 to 20 carbon atoms such as a vinyl group, an allyl group, a propenyl group, an isopropenyl group, a butenyl group, an isobutenyl group, a 1, 3-butadienyl group, a pentenyl group, a hexenyl group and the like.

In formula (9) described above, examples of the alkynyl group represented by R¹ include linear or branched alkynyl groups having 2 to 20 carbon atoms such as an ethynyl group, a propynyl group, a butynyl group, a pentynyl group, a hexynyl group and the like.

In formula (9) described above, examples of the aryl group represented by R³ include monocyclic or polycyclic aromatic hydrocarbon groups having 6 to 20 carbon atoms. Specific examples of the aryl group include a phenyl group, a naphthyl group, an anthryl group, a phenanthryl group, a pyrenyl group and the like.

In formula (9) described above, examples of the heteroaryl group represented by R³ include monocyclic or polycyclic aromatic heterocyclic groups having 2 to 9 ring carbon atoms and containing 1 to 4 heteroatoms selected from an oxygen atom, a sulfur atom and a nitrogen atom. Specific examples of the heteroaryl group include a pyrrolyl group, a pyridyl group, an imidazolyl group, a pyrazolyl group, a pyrazinyl group, a pyridazinyl group, a pyrimidinyl group, a triazolyl group, a tetrazolyl group, a furanyl group, a thienyl group, an oxazolyl group, an isoxazolyl group, an oxadiazolyl group, a thiazolyl group, an isothiazolyl group, a thiadiazolyl group, an indolyl group, an isoindolyl group, a benzimidazolyl group, an indazolyl group, a benzotriazolyl group, a tetrahydroquinolyl group, a quinolyl group, a tetrahydroisoquinolyl group, an isoquinolyl group, a quinolizinyl group, a cinnolinyl group, a phthalazinyl group, a quinazolinyl group, a quinoxalinyl group, a naphthyridinyl group, a pyrrolopyridyl group, an imidazopyridyl group, a pyrazolopyridyl group, a pyridopyrazyl group, a purinyl group, a pteridinyl group, a benzofuranyl group, an isobenzofuranyl group, a benzothienyl group, a benzoxazolyl group, a benzoisoxazolyl group, a benzoxadiazolyl group, a benzothiazolyl group, a benzoisothiazolyl group, a benzothiadiazolyl group, a thiazolopyridyl group and the like.

In formula (9) described above, examples of the arylalkyl group represented by R″ include the alkyl group having 1 to 6 carbon atoms substituted with the aryl group described above. Specific examples of the arylalkyl group include a benzyl group, a phenethyl group, a 3-phenylpropyl group, a 4-phenylbutyl group, a 1-phenylethyl group, a 2-phenylpropan-2-yl group and the like.

In formula (9) described above, examples of the heteroarylalkyl group represented by R³ include the alkyl group having 1 to 6 carbon atoms substituted with the hetercaryl group described above. Specific examples of the heteroarylalkyl group include a pyridylmethyl group, a pyridylethyl group, an imidazolylmethyl group, an imidazolylethyl group, a pyrazolylmethyl group, a pyrazolylethyl group, a pyrazinylmethyl group, a pyrazinylethyl group, a pyridazinylmethyl group, a pyridazinylethyl group, a pyrimidinylmethyl group, a pyrimidinylethyl group, an oxazolylmethyl group, an oxazolylethyl group, a thiazolylmethyl group, a thiazolylethyl group and the like.

Examples of a substituent which may be included in the aryl group, the alkenyl group, the alkynyl group, the heteroaryl group, the arylalkyl group or the heteroarylalkyl group described above include an alkyl group, an alkenyl group, an alkynyl group, an aryl group, a heteroaryl group, an alkoxy group, an alkoxycarbonyl group, a halogen atom, a hydroxy group, a carboxy group, an amino group, a nitro group, a nitroxy group, a mercapto group, a cyanate group, a thiocyanate group, an isothiocyanate group, a sulfo group, a sulfamino group, a sulfino group, a sulfamoyl group, a phospho group, a phosphono group, a boronyl group, a cyano group and the like. The number of substituents is not particularly limited.

Specific examples of the Weinreb amide represented by formula (9) described above are shown below. However, the Weinreb amide in the present embodiment is not limited to these examples. For convenience, only E isomers are shown below, but the cis-trans isomer mixture according to the present embodiment also includes 2 isomers of the following substances.

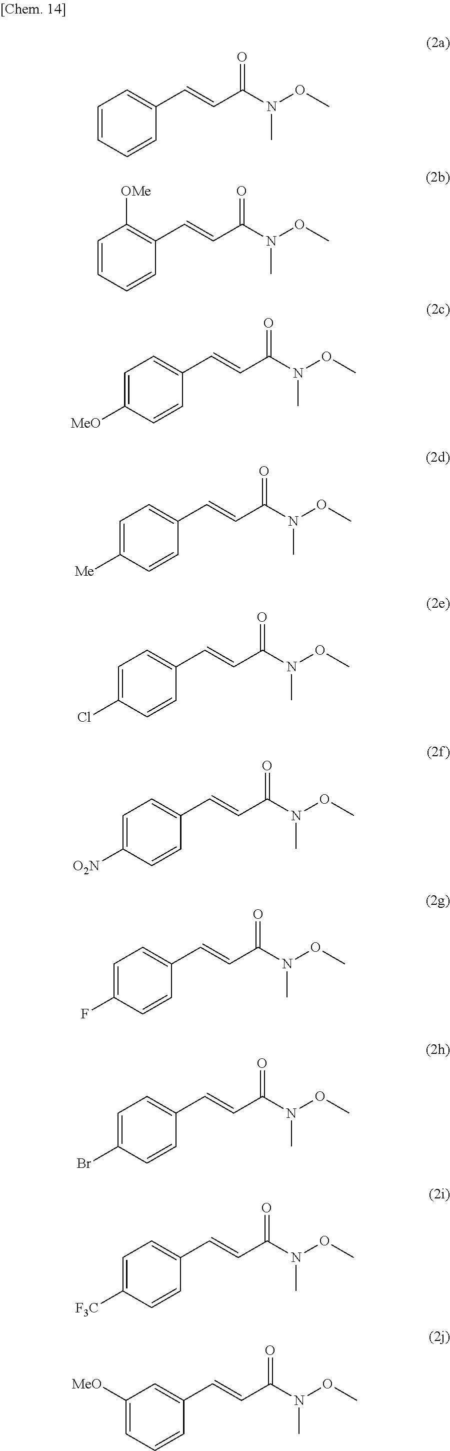

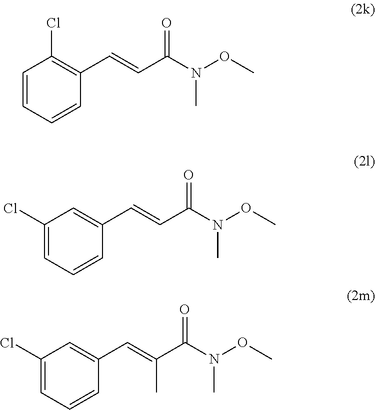 [SMILES: CON(C)C(=O)/C=C/c1ccccc1Cl ; CON(C)C(=O)/C=C/c1cccc(Cl)c1 ; CON(C)C(=O)/C(C)=C/c1cccc(Cl)c1]

### (Step A)

In the step A, the cis-trans isomer mixture is separated into one diastereomer and the other diastereomer by high performance liquid chromatography (HPLC) using the separation column.

The cis-trans isomer mixture is not particularly limited as long as Z and E isomers are included. The isomer excess ratio of the cis-trans isomer mixture (the ratio (%) of one diastereomer—the ratio (%) of the other diastereomer) may be 0% to 25%, 0% to 10% or 0% to 5%.

The separation column for separating the cis-trans isomers is not particularly limited, and any separation column which is used in high performance liquid chromatography (HPLC) can be adopted. For example, a normal phase column utilizing a silanol group is preferably adopted.

### (Step B)

In the step B, light is applied to the other diastereomer obtained in the step A or the step C to be described later in the presence of the photosensitizer in the photoisomerization reactor to induce the photoisomerization reaction, and thus the cis-trans isomer mixture is generated from the diastereomer. As will be described later, light having a specific wavelength is applied to the other diastereomer to induce the photoisomerization reaction.

The other diastereomer to which the light is applied is in a state where the other diastereomer is dissolved in a solvent. In other words, the light can be applied to an eluate which is eluted in the step A and contains the other diastereomer.

In the step B, the solution containing the other diastereomer may include a solvent. The solvent is not particularly limited, and examples thereof include one or two or more solvents selected from the group consisting of methanol (MeOH), ethanol (EtOH), isopropanol (IPA), diethyl ether (Et₂O), ethyl acetate (AcOEt), tetrahydrofuran (THF), dimethyl sulfoxide (DMSO), acetonitrile (MeCN), N, N-dimethylformamide (DMF) and dichloromethane (CH₂Cl₂). The solvent may be obtained by mixing two or more solvents, or may be mixed with water (H₂O) or hexane (Hex).

In the step B, as the solvent for dissolving the other diastereomer, one or two or more solvents selected from the group consisting of methanol (MeOH), dimethyl sulfoxide (DMSO), dichloromethane (CH₂Cl₂) and ethanol (EtOH) is preferable, and a mixed solvent of dichloromethane (CH₂Cl₂) and methanol (MeOH) (mixed solvent in which the mixing ratio (volume ratio) of CH₂Cl₂: MeOH is 1:1 to 9:1) or methanol (MeOR) is more preferably used. The solvent described above is used, and thus it is possible to enhance the reaction rate of the photoisomerization reaction in the step B.

The wavelength of the light applied in the step B is preferably adjusted as necessary depending on, for example, the types or the like of cis-trans isomer mixture and photosensitizer, and may be, for example, 200 nm to 450 nm, 200 nm to 400 nm or 254 nm to 365 nm. Although the time during which the light is applied differs depending on the type of cis-trans isomer mixture, the wavelength of the light applied or the like, in general, it is possible to sufficiently induce the photoisomerization reaction by applying the light for 5 minutes to 2 hours.

### (Step C)

In the step C, the cis-trans isomer mixture obtained in the step B is separated into one diastereomer and the other diastereomer. The isomer excess ratio of the cis-trans isomer mixture (the ratio (%) of one diastereomer—the ratio (*) of the other diastereomer) may be 0% to 25%, 0% to 10% or 0% to 5%.

As the separation method in the step C, the same high performance liquid chromatography method using the separation column as in the step A is adopted.

The steps B and C described above are performed, and thus as compared with a case where only the step A is performed, it is possible to significantly enhance the yield of the desired diastereomer. The steps B and C described above are repeated as necessary, and thus it is also possible to further enhance the yield of the desired diastereomer.

### (Step D)

The step D is performed in a stage preceding the step A. In the step D, when only the other diastereomer of Z and E isomers is used as a starting material, light is applied to a solution containing only the other diastereomer in the presence of a photosensitizer in the photoisomerization reactor to induce a photoisomerization reaction, and thus a cis-trans isomer mixture is generated. The other diastereomer to which the light is applied is in a state where the other diastereomer is dissolved in a solvent. The conditions of application of light in the step D are the same as in the step B.

### [Photosensitizer]

In the step B, light is applied in the presence of the photosensitizer to be able to enhance the efficiency of the photoisomerization reaction. The photosensitizer includes a thioxanthone skeleton represented by formula (1) below.

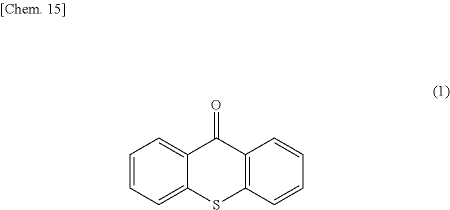 [SMILES: O=c1c2ccccc2sc2ccccc12]

The photosensitizer is preferably a solid-phase photosensitizer which is covalently solid-phased to a carrier such as silica gel or the like. The solid-phase photosensitizer is used to easily separate the cis-trans isomer mixture obtained in the step B and the photosensitizer. The solid-phase photosensitizer is preferably a solid-phase photosensitizer in which a structure represented by formula (2) below is covalently solid-phased to a carrier such as silica gel or the like via a linker.

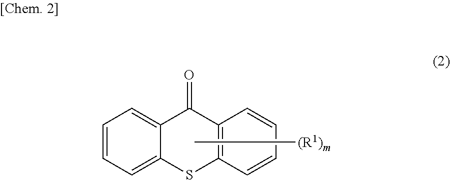

[In formula (2), R¹ represents an alkyl group having 2 to 3 carbon atoms, a hydroxy group or an alkoxycarbonyl group, m represents an integer from 0 to 5 and when m is an integer from 2 to 5, a plurality of Ris may be the same as or different from each other.]

In formula (2) described above, examples of the alkyl group represented by R¹ include an ethyl group, an n-propyl group, an isopropyl group and the like. In formula (2) described above, examples of the alkoxycarbonyl group represented by R¹ include a group which has a linear or branched alkyl moiety having 1 to 3 carbon atoms.

The photosensitizer is covalently solid-phased to the carrier via the linker, and thus it is preferably possible to suppress the leakage of the photosensitizer into a reaction system. The linker preferably includes a functional group which includes an alkylene group, an arylene group, an —NR—C (O)— group (where R independently represents a hydrogen atom, an alkyl group or a substituted alkyl group) or a combination thereof.

The solid-phase photosensitizer includes, for example, a structure represented by formula (3) below.

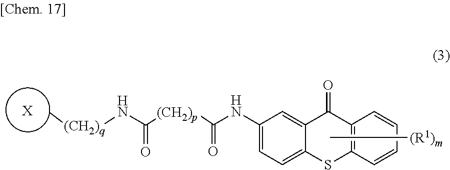

[In formula (3), R¹ and m are as defined in formula (2) above, p represents an integer from 1 to 10, q represents an integer from 2 to 10 and X represents a carrier.]

In formula (3) described above, p is preferably an integer from 1 to 8, and more preferably an integer from 1 to 6. Here, q is preferably an integer from 2 to 8, and more preferably an integer from 2 to 6.

As the carrier to which the photosensitizer is solid-phased, a carrier formed of any material such as silicon, glass a polymeric material or the like can be adopted. The carrier may be a composite material of the materials described above. Examples of the carrier include silica gel. The particle diameter of the carrier is preferably 60 to 100 μm.

Method for manufacturing solid-phase photosensitizer

A method for manufacturing a solid-phase photosensitizer including the structure represented by formula (3) above includes: a step of reacting a compound represented by formula (5) below with a dicarboxylic acid represented by formula (6) below or an anhydride thereof to obtain a compound represented by formula (7) below; and a step of reacting a carrier including a structure represented by formula (8) below with a compound represented by formula (7) below to obtain a solid-phase photosensitizer including the structure represented by formula (3) above.

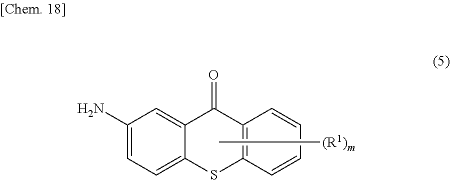

[In formula (5), R¹ and m are as defined in formula (3) above.]

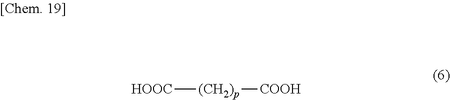

[In formula (6), p is as defined in formula (3) above.]

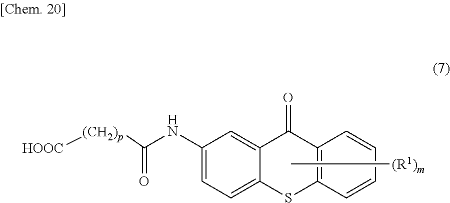

[In formula (7), R¹, m and p are as defined in formula (3) above.]

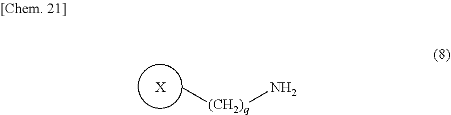

[In formula (8), q and X are as defined in formula (3) above.]
<Other photosensitizers>

Examples of a photosensitizer other than those described above which can be applied to the diastereomer preparation method according to the present embodiment include a compound represented by formula (4) below. The compound represented by formula (4) below may be solid-phased to a carrier.

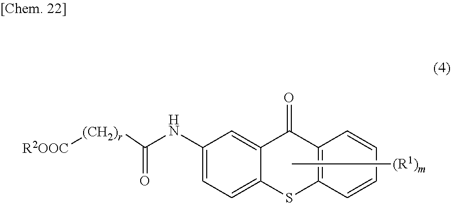

[In formula (4), R¹ represents an alkyl group having 2 to 3 carbon atoms, a hydroxy group or an alkoxycarbonyl group, m represents an integer from 0 to 5 and when m is an integer from 2 to 5, a plurality of R¹s may be the same as or different from each other, r represents an integer from 1 to 7 and R² represents a hydrogen atom or an alkyl group having 1 to 4 carbon atoms.] [00€7]

### <Recycling HPLC>

The recycling HPLC according to the present embodiment is recycling HPLC used in the diastereomer preparation method described above, and includes: a separation column which separates a cis-trans isomer mixture into one diastereomer and the other diastereomer; and a photoisomerization reactor which applies light to the other diastereomer obtained in the separation column to induce the photoisomerization reaction of the diastereomer.

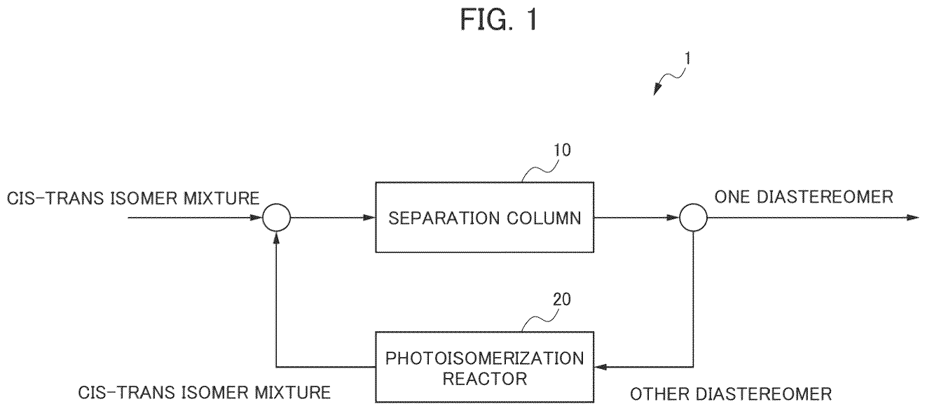

An example of a schematic configuration of the recycling HPLC according to the present embodiment is shown in FIG. 1. As shown in FIG. 1, the recycling HPLC 1 includes a separation column 10 and a photoisomerization reactor 20.

The separation column 10 separates a cis-trans isomer mixture into one diastereomer (for example, a 2 isomer) and the other diastereomer (for example, an E isomer). As the separation column 10, any separation column used in the high performance liquid chromatography method can be adopted.

The photoisomerization reactor 20 applies light to the other diastereomer (for example, an E isomer) obtained in the separation column 10 in the presence of the photosensitizer to induce the photoisomerization reaction of the diastereomer, and thereby generates the cis-trans isomer mixture. The light source of the photoisomerization reactor 20 is not particularly limited as long as the light of a wavelength necessary for inducing the photoisomerization reaction of the other diastereomer is applied. Specific examples of the light source include a high-pressure mercury lamp, a low-pressure mercury lamp, a xenon lamp, an LED lamp and the like. When a material which absorbs the light from the light source or a material which changes the wavelength of the light from the light source is interposed between the light source and the other diastereomer, it is preferable to apply the light with consideration given to the absorption of the light or a change in the wavelength of the light. The photoisomerization reactor 20 feeds the cis-trans isomer mixture obtained to the separation column 10 again.

In the photoisomerization reactor 20, for example, a flow path having optical transparency such as a glass column or the like is filled with the solid-phase photosensitizer described above, and the light is applied from the light source to the other diastereomer (for example, an E isomer) flowing through the flow path. The flow path may be filled with optically transparent particles in addition to the solid-phase photosensitizer. In this way, it is possible to cause the light applied from the light source to fully spread to the center of the flow path. It is also possible to reduce the amount of solid-phase photosensitizer used, and to reduce the risk of leakage of the solid-phase photosensitizer. The optically transparent particles are not particularly limited, and examples thereof include glass beads, irregular glass powder, resin beads and the like. The median diameter (D50) of the optically transparent particles can be, for example, about 10 to 100 μm. The mass ratio of the solid-phase photosensitizer to the optically transparent particles filled in the flow path is preferably 1 to 50% by mass with respect to the total mass of the solid-phase photosensitizer and the optically transparent particles, more preferably 5 to 25% by mass and further preferably 5 to 10% by mass.

As described above, with the recycling HPLC 1, it is possible to significantly enhance the yield of the desired diastereomer by repeating optical splitting and photo-racemization in the separation column 10 and the photoisomerization reactor 20. The recycling HPLC 1 which is a circulation-type system is adopted, and thus it is possible to reuse the solvent and to easily repeat the separation of diastereomers and the photoisomerization reaction.

### EXAMPLES

Although the present invention will be more specifically described below using Examples, the present invention is not limited to these Examples.

<Preparation of solid-phased thioxanthone>

(1) Synthesis of 4-oxo-4-[(9-oxo-9H-thioxante-2-nyl) amino]butanoic acid (formula 7 below)

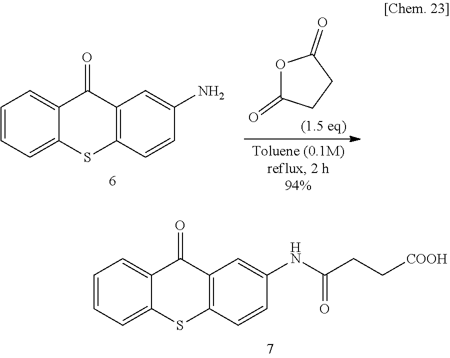 [SMILES: Nc1ccc2sc3ccccc3c(=O)c2c1 ; O=C1CCC(=O)O1 ; O=C(O)CCC(=O)Nc1ccc2sc3ccccc3c(=O)c2c1]

Into a 100 ml recovery flask, 544 mg (2.4 mmol, 1.0 eq) of 4-aminothioxanthone (formula 6 above) and 360 mg (3.6 mmol, 1.5 eq) of succinic acid were added, were dissolved in toluene (24 mb, 0.1 M) under an argon flow and were heated to reflux for 2 hours. The mixture was returned to room temperature and washed with toluene, and a target compound (formula 7 above) was filtered out as a yellow solid (yield of 738 mg and 94%).

(2) Solid phasing of 4-oxo-4-[(9-oxo-9H-thioxante-2-nyl) amino]butanoic acid: synthesis of solid-phased thioxanthone (formula 8 below)

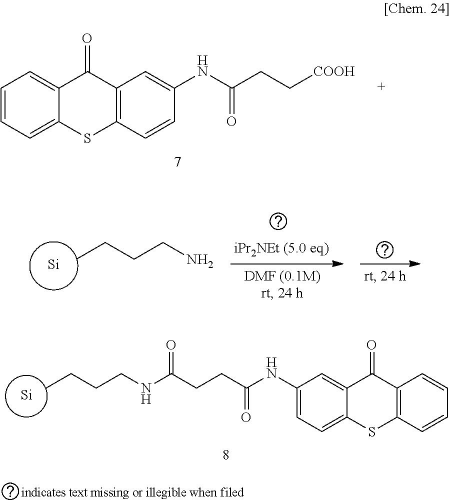

Into a 50 mL recovery flask, 1 g (0.6-1.3 mmol, 1.0 eq) of 3-aminopropyl silica gel, 426 mg (1.3 mmol, 1.0 eq) of 4-oxo-4-((9-oxo-9H-thioxante-2-nyl) amino]butanoic acid (formula 7 above), 1.35 g (2.6 mmol, 2.0 eq) of 1H-benzotriazol-1-yloxytripyrrolidinophosphonium hexafluorophosphate and 1.1 mb (6.5 mmol, 5.0 eq) of N-ethyldiisopropylamine were dissolved in DMF (13 mL, 0.1 M), and the mixture was stirred at room temperature for 24 hours. The solid phase was then filtered out, was washed sufficiently with dichloromethane and acetonitrile and was then dried. After drying, the solid phase was added to 10 mL of a mixed solvent of pyridine: acetic anhydride=9:1, and was stirred at room temperature for 24 hours. The solid phase was then filtered out, was washed sufficiently with dichloromethane and was then dried. After drying, a covalent solid-phase photosensitizer (formula 8 above) was obtained as a yellow solid.

(Photoisomerization reaction test)

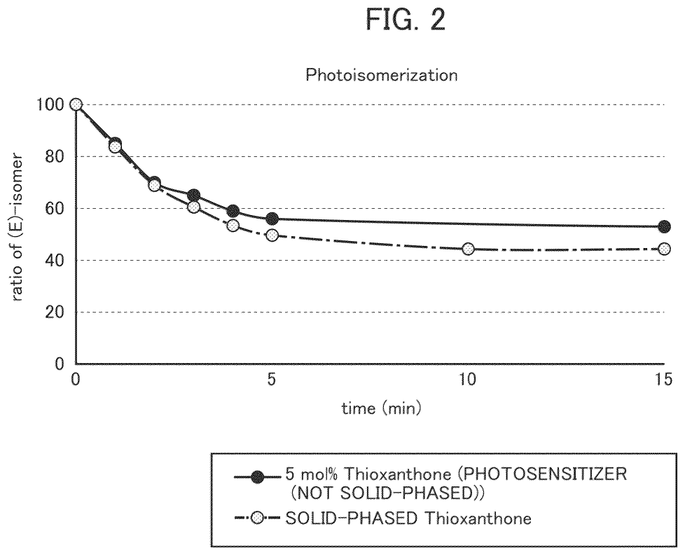

FIG. 2 is a diagram showing respective changes over time in the abundance ratio of the other diastereomer when a photoisomerization reaction is performed using thioxanthone serving as a photosensitizer without the thioxanthone being solid-phased to a carrier (5 mol % thioxanthone) and when a photoisomerization reaction is performed using the solid-phased thioxanthone prepared as described above. As the target of the photoisomerization reaction, (E)-cinnamamide was used, and the applied light was set to 405 nm. As shown in FIG. 2, it is clear that the solid-phased thioxanthone was used, and thus the photoisomerization reaction was preferably performed as compared with a case where thioxanthone was used without being solid-phased to a carrier.

(Leakage test)

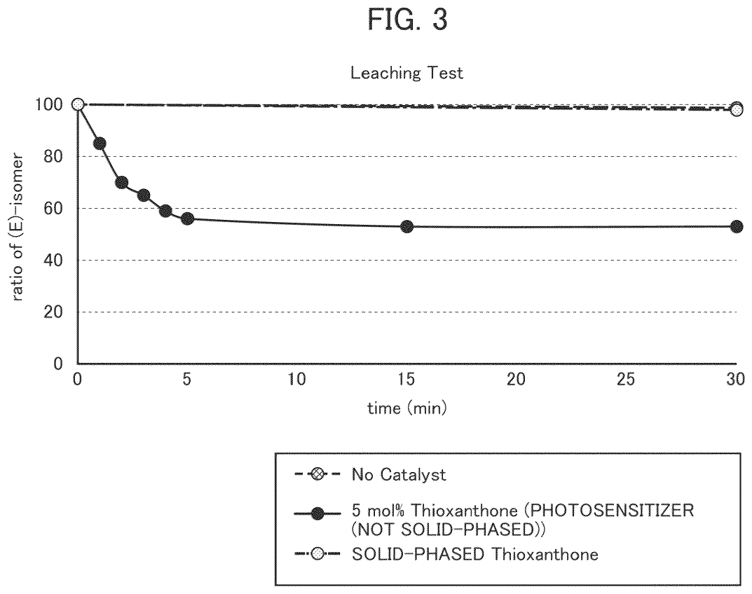

FIG. 3 is a diagram showing the results of a test for checking whether isomerization occurred, that is, whether a photosensitizer flowed out of the system, by stirring the photosensitizer in a solvent for a certain period of time, adding (E)-cinnamamide to the filtrate (the solvent from which the photosensitizer was removed) and applying light when thioxanthone serving as a photosensitizer was used without being solid-phased to a carrier (5 mol % thioxanthone), when a photosensitizer was not used (No Catalyst) and when the solid-phased thioxanthone prepared as described above was used. As shown in FIG. 3, it is clear that the solid-phased thioxanthone was used, and thus the photosensitizer did not flow out of the system.

(Calculation of required amount of ultraviolet light applied)

The solid-phased thioxanthone prepared as described above was used, and thus the required amount of ultraviolet light applied (J/cm2) was calculated. In the calculation, the amount of light (J/cm2) required until the abundance ratio of an E diastereomer reached 55% (the abundance ratio of a Z diastereomer reached 45%) was calculated by applying light of 405 nm (LED) to each of E diastereomers (solvent: 0.01M of acetonitrile) represented by formulae below in the presence of 5 mol % of the photosensitizer. The results are shown in Table 1.

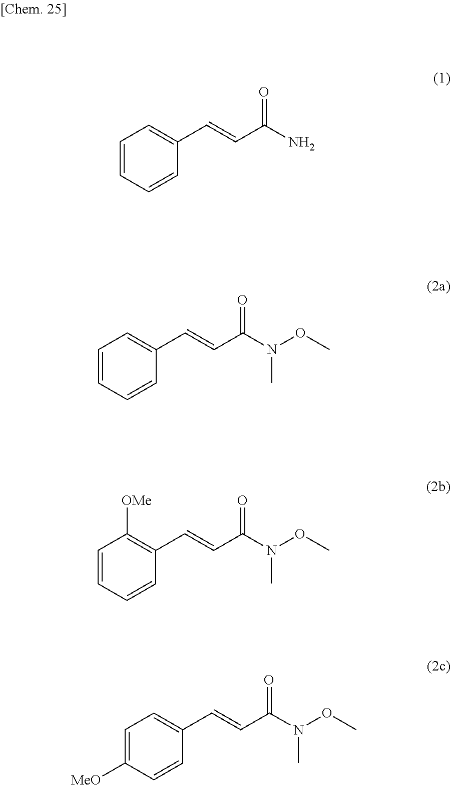 [SMILES: NC(=O)/C=C/c1ccccc1 ; CON(C)C(=O)/C=C/c1ccccc1 ; COc1ccccc1/C=C/C(=O)N(C)OC ; COc1ccc(/C=C/C(=O)N(C)OC)cc1]

TABLE 1
Required amount of ultraviolet light applied (J/cm²)

| | |
|---|---|
| (1) | 16.638 |
| (2a) | 16.638 |
| (2b) | 24.871 |
| (2c) | 12.222 |

### Reference Example: Preparation of Photosensitizer

As with the solid-phased thioxanthones described above, each of photosensitizers in a state where the photosensitizers were not solid-phased to a carrier were prepared as shown in formulae below.

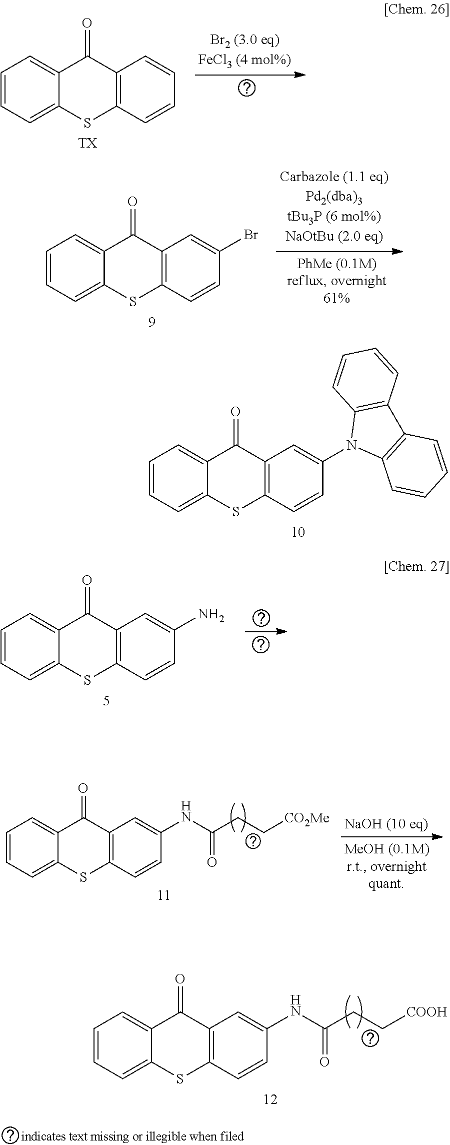

(Calculation of required amount of ultraviolet light applied)

Each of the photosensitizers prepared as described above was used, and thus the required amount of ultraviolet light applied (J/cm2) was calculated. In the calculation, the amount of light (J/cm2) required until the abundance ratio of (E)-cinnamamide reached 55% (the abundance ratio of (Z)-cinnamamide reached 45%) was calculated by applying light of 405 nm (LED) to (E)-cinnamamide (solvent: 0.01M of acetonitrile) in the presence of 5 mols of the photosensitizer. The results are shown in Table 2.

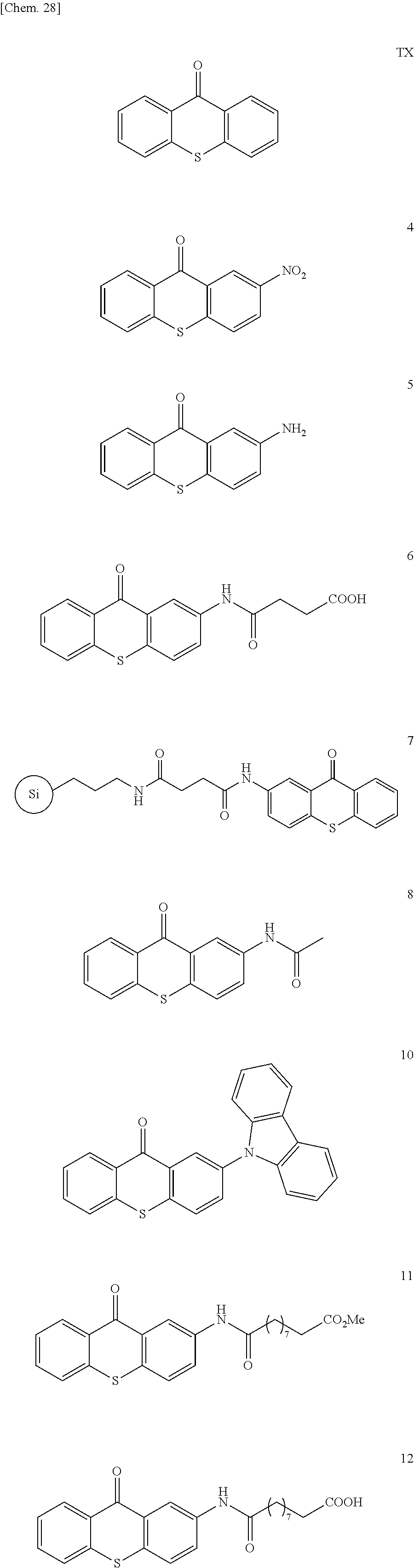

TABLE 2
Required amount of ultraviolet light applied (J/cm²)

| | |
|---|---|
| TX | 12.372 |
| 4 | 6.805 |
| 5 | 150.033 |
| 6 | 4.385 |
| 7 | 4.379 |
| 8 | 4.131 |
| 10 | 7.854 |
| 11 | 3.459 |
| 12 | 6.688 |

As shown in Table 2, it is clear that as compared with thioxanthone (TX), for the photosensitizer 6 to which the linker was coupled and the solid-phase photosensitizer 7, the required amount of ultraviolet light applied was lowered. It is also clear that an amide bond included in thioxanthone reduced the required amount of ultraviolet light applied.

### Example 1: Selective Preparation of Cinnamamide Utilizing Recycling HPLC

5.7 g of fuji glass beads (FGB-200) and 300 mg of a covalent bond type photosensitizer which was the solid-phased thioxanthone prepared as described above were mixed such that a total amount was 6 g and the abundance ratio was 95:5, and the mixture was filled in a glass column [YMC CO., LTD, ECOPLUS (+5 mm)] such that a total length was 210 mm. The glass column was connected to a Multiple Preparative HPLC Forte and was set vertically, and a solution was passed through the glass column at a flow rate of 1.0 mb/min with a solvent of 100% of acetonitrile. Then, the glass column was coupled to a YMC-Pack SIL-06 (250×20.0 mml. D.S-5 μm, 6 nm). The glass column was placed in a photoisomerization reactor (made by IWASAKI ELECTRIC Co., Ltd.), and the solution was passed through the glass column at a flow rate of 4.7 ml/min with a solvent of 100% of acetonitrile.

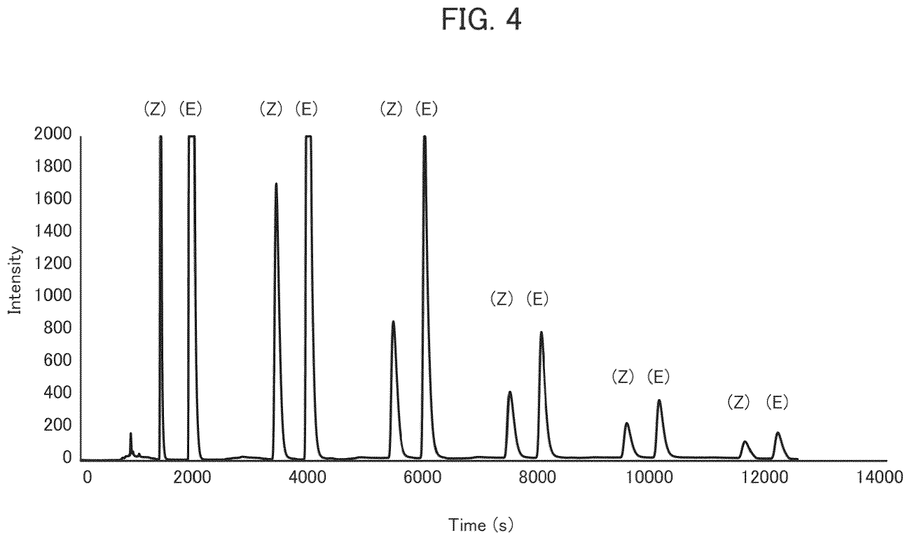

Then, a sample was prepared by dissolving 10 mg (0.0679 mmol) of (E)-cinnamamide in 1 mb of acetonitrile. A cooling fan attached to the photoisomerization reactor was turned on, a direct-current stabilized power supply was turned on at a current value of 1.0 A to apply light of 405 nm and thereafter the entire amount of sample was added. After the addition, the application was completed when five minutes elapsed. A detected (Z)-cinnamamide peak was collected, and the application of light was started again at the same time when recycling of (E)-cinnamamide was started. The application was completed when five minutes elapsed after the completion of the recycling. The step described above was repeated six times. A relationship between the peak intensity of Z and E isomers and the elapsed time after the start of recycling is shown in the graph of FIG. 4.

By performing the isomerization of (E)-cinnamamide with recycling RPLC in the presence of solid-phased thioxanthone, (Z)-cinnamamide was obtained in a yield of 95% with a purity of Z/E ratio=99:1, with the result that it was confirmed that the photoisomerization reaction was performed efficiently.

### Example 2: Selective Preparation of (2E)-N-Methoxy-3-(4-Methoxyphenyl)-N-Methyl2-Propenamide Utilizing Recycling HPLC

As in Example 1, the recycling HPLC was set using the solid-phased thioxanthone prepared as described above.

Then, a sample was prepared by dissolving 10 mg (0.0679 mmol) of (2c-E) shown in formula below: (2E)-N-methoxy-3-(4-methoxyphenyl)-N-methyl2-propenamide in 1 mL of acetonitrile. The cooling fan attached to the photoisomerization reactor was turned on, the direct-current stabilized power supply was turned on at a current value of 1.0 A to apply light of 405 nm and thereafter the entire amount of sample was added. After the addition, the application was completed when five minutes elapsed. A detected (2c-z) shown in formula above: (2%)-N-methoxy-3-(4-methoxyphenyl)-N-methyl2-propenamide peak was collected, and the application of light was started again at the same time when recycling of (2c-E) was started. The application was completed when five minutes elapsed after the completion of the recycling. The step described above was repeated six times.

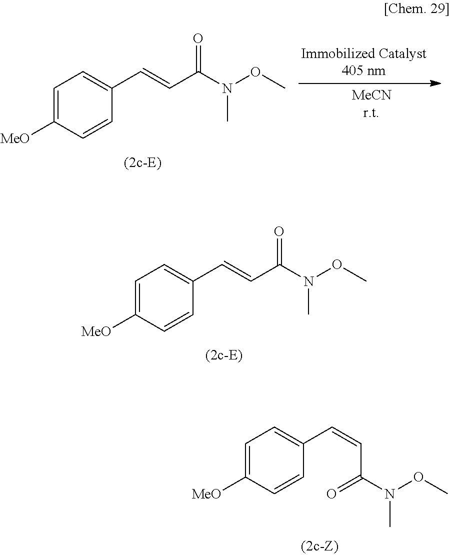 [SMILES: COc1ccc(/C=C/C(=O)N(C)OC)cc1 ; COc1ccc(/C=C/C(=O)N(C)OC)cc1 ; COc1ccc(/C=C\C(=O)N(C)OC)cc1]

By performing the isomerization of (2c-E) in formula above with the recycling HPLC in the presence of solid-phased thioxanthone, (2c-2) in formula above was obtained in a yield of 60% with a purity of Z/E ratio=>99:1, with the result that it was confirmed that the photoisomerization reaction was performed efficiently.

### <Relationship Between Solvent and Reaction Rate in Step B>

(E)-cinnamamide (3.0 mg, 0.02 mmol) was dissolved in each of solvents (0.01 M) shown in Tables 3 and 4 below, and thus solutions were prepared. Light of 405 nm (LED, 18.1 mW/cm2) was applied to the solvents in the presence of thioxanthone (5 mols) serving as a photosensitizer, and a time (ti/z(s)) required until the abundance ratio of (E)-cinnamamide reached 50% (the abundance ratio of (Z)-cinnamamide reached 50%) was calculated. The rate constant (k> (M-IS-1)) at that time was determined. The results are shown in Tables 3 and 4. The abbreviations of the solvents shown in Tables 3 and 4 have the same meanings as those shown in the embodiment described above, and the ratios of the solvents mean the mixing ratios (volume ratio).

TABLE 3

| | | | Rate |
|---|---|---|---|
| | | Time t_{1/2} | constant k₂ |
| Entry | Solvent | (s) | (M⁻¹S⁻¹) |
| 1 | MeCN | 960 | 0.72 |
| 2 | CH₂Cl₂ | 560 | 1.24 |
| 3 | MeOH | 251 | 2.76 |
| 4 | CH₂Cl₂:MeOH = 9:1 | 280 | 2.47 |
| 5 | CH₂Cl₂:MeOH = 4:1 | 306 | 2.26 |
| 6 | CH₂Cl₂:MeOH = 2:1 | 173 | 3.99 |
| 7 | CH₂Cl₂:MeOH = 1:1 | 268 | 2.58 |
| 8 | CH₂Cl₂:MeOH = 1:2 | 563 | 1.23 |
| 9 | CH₂Cl₂:MeOH = 1:4 | 617 | 1.12 |
| 10 | CH₂Cl₂:MeOH = 1:9 | 636 | 1.09 |

TABLE 4

| | | | Rate |
|---|---|---|---|
| | | Time t_{1/2} | constant k₂ |
| Entry | Solvent | (s) | (M⁻¹S⁻¹) |
| 11 | AcOEt | 1946 | 0.36 |
| 12 | THF | 1500 | 0.46 |
| 13 | iPA | 807 | 0.86 |
| 14 | EtOH | 657 | 1.06 |
| 15 | DMSO | 363 | 1.91 |
| 16 | Et₂O | 3971 | 0.17 |
| 17 | DMF | 725 | 0.96 |
| 18 | EtOH:Hex = 5:1 | 643 | 1.08 |
| 19 | iPA:Hex = 5:1 | 804 | 0.86 |
| 20 | MeOH:Hex = 5:1 | 354 | 1.96 |
| 21 | MeOH:H₂O = 5:1 | 541 | 1.27 |
| 22 | EtOH:H₂O = 5:1 | 531 | 1.31 |
| 23 | AcOEt:Hex = 5:1 | 2707 | 0.26 |
| 24 | Et₂O:Hex = 5:1 | 1472 | 0.47 |

It is clear from the results shown in Tables 3 and 4 that: the reaction rate of the photoisomerization reaction can be enhanced by using one or two or more solvents selected from the group consisting of methanol (MeOH), dimethyl sulfoxide (DMSO), dichloromethane (CH₂Cl₂) and ethanol (EtOH) as the solvent contained in the solution in step B. It is clear from the viewpoint described above that the solvent is more preferably a mixed solvent of dichloromethane (CH₂C12) and methanol (MeOH) (a mixed solvent in which the mixing ratio (volume ratio) of CH₂Cl>: MeOH is 1:1 to 9:1) ox methanol (MeOH).

### EXPLANATION OF REFERENCE NUMERALS

1: recycling BPLC, 10: separation column, 20: photoisomerization reactor

1. A diastereomer preparation method for selectively preparing one diastereomer of cis-trans isomers by utilizing recycling HPLC capable of continuously circulating through a separation column that separates the cis-trans isomers and a photoisomerization reactor that induces a photoisomerization reaction, the diastereomer preparation method comprising:
step A: a separation step of introducing a cis-trans isomer mixture into the separation column to isolate the one diastereomer;
step B: a photoisomerization reaction step of applying light to a solution containing the other diastereomer after the step A or step C in presence of a photosensitizer in the photoisomerization reactor to induce the photoisomerization reaction; and
step C: a step of introducing the cis-trans isomer mixture generated in the step B into the separation column to isolate the one diastereomer,
wherein the photosensitizer includes a thioxanthone skeleton represented by formula (1) below:

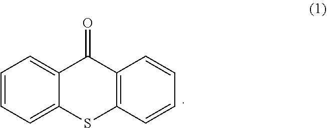 [SMILES: O=c1c2ccccc2sc2ccccc12]

2. The diastereomer preparation method according to claim 1, wherein the photosensitizer is a solid-phase photosensitizer in which a structure represented by formula (2) below is covalently solid-phased to a carrier in the photoisomerization reactor via a linker: 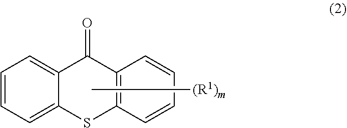
wherein R¹ represents an alkyl group having 2 to 3 carbon atoms, a hydroxy group, or an alkoxycarbonyl group, m represents an integer from 0 to 5, and when m is an integer from 2 to 5, a plurality of R's may be the same as or different from each other.

3. The diastereomer preparation method according to claim 2, wherein the linker includes an alkylene group, an arylene group, an —NR—C(O)— group in which R independently represents a hydrogen atom, an alkyl group, or a substituted alkyl group, or a functional group including a combination thereof.

4. The diastereomer preparation method according to claim 3, wherein the solid-phase photosensitizer includes a structure represented by formula (3) below: 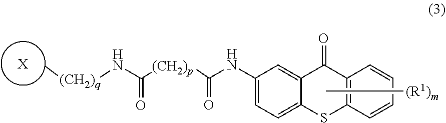
wherein R¹ and m are as defined in formula (2) above, p represents an integer from 1 to 10, q represents an integer from 2 to 10, and X represents a carrier.

5. The diastereomer preparation method according to claim 1, wherein the photosensitizer is represented by formula (4) below: 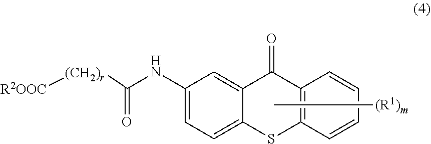
wherein R¹ represents an alkyl group having 2 to 3 carbon atoms, a hydroxy group, or an alkoxycarbonyl group, m represents an integer from 0 to 5, and when m is an integer from 2 to 5, a plurality of R¹s may be the same as or different from each other, r represents an integer from 1 to 7, and R² represents a hydrogen atom or an alkyl group having 1 to 4 carbon atoms.

6. The diastereomer preparation method according to claim 2, wherein a material of the carrier is silica, glass, a polymer material, or a composite material thereof.

7. The diastereomer preparation method according to claim 1, wherein the cis-trans isomer is a cis-trans isomer of an alkene including an amide group in a molecule.

8. The diastereomer preparation method according to claim 7, wherein the alkene including an amide group in the molecule is a Weinreb amide.

9. A solid-phase photosensitizer comprising a structure represented by formula (3) below: 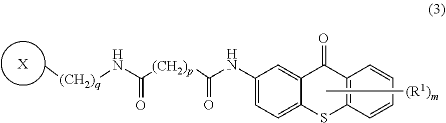
wherein R¹ represents an alkyl group having 2 to 3 carbon atoms, a hydroxy group, or an alkoxycarbonyl group, m represents an integer from 0 to 5, and when m is an integer from 2 to 5, a plurality of R¹s may be the same as or different from each other, p represents an integer from 1 to 10, q represents an integer from 2 to 10, and X represents a carrier.

10. A method for manufacturing a solid-phase photosensitizer including a structure represented by formula (3) below, the method comprising:
a step of reacting a compound represented by formula (5) below with a dicarboxylic acid represented by formula (6) below or an anhydride thereof to obtain a compound represented by formula (7) below; and
a step of reacting a carrier including a structure represented by formula (8) below with a compound represented by formula (7) below to obtain a solid-phase photosensitizer including a structure represented by formula (3) below: 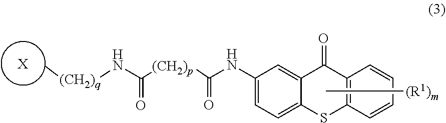
wherein R¹ represents an alkyl group having 2 to 3 carbon atoms, a hydroxy group, or an alkoxycarbonyl group, m represents an integer from 0 to 5, and when m is an integer from 2 to 5, a plurality of R¹s may be the same as or different from each other, p represents an integer from 1 to 10, q represents an integer from 2 to 10, and X represents a carrier; 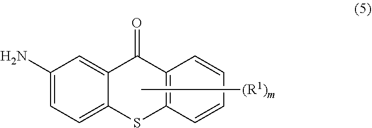
wherein R¹ and m are as defined in formula (3) above; 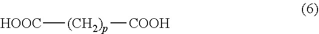
wherein p is as defined in formula (3) above; 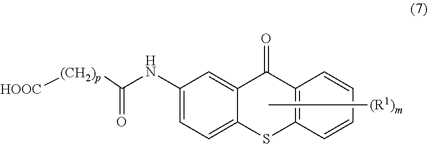
wherein R¹, m and p are as defined in formula (3) above; 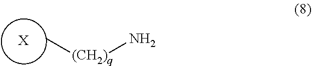
wherein q and X are as defined in formula (3) above.

11. A compound represented by formula (4) below: 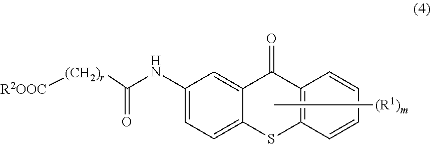
wherein R¹ represents an alkyl group having 2 to 3 carbon atoms, a hydroxy group, or an alkoxycarbonyl group, m represents an integer from 0 to 5, and when m is an integer from 2 to 5, a plurality of R¹s may be the same as or different from each other, r represents an integer from 1 to 7, and R² represents a hydrogen atom or an alkyl group having 1 to 4 carbon atoms.
